(19) United States
(12) Patent Application Publication
(10) Pub. No.: US 2004/0058868 A1
(43) Pub. Date: Mar. 25, 2004

(54) Methods for identification of compounds modulating insulin resistance

(76) Inventors: Stephen James, Vallingby (SE); Christina Kaiser, Ronninge (SE)

Correspondence Address: FISH & RICHARDSON PC, 225 FRANKLIN ST, BOSTON, MA, 02110, US

(21) Appl. No.: 10/615,252

(22) Filed: Jul. 8, 2003

(30) Foreign Application Priority Data
Jul. 9, 2002 (SE) 0202157-4

(51) Int. Cl. A61K 48/00; A61K 38/12; A61K 38/17; A61K 31/198; A61K 31/19

(52) U.S. Cl. 514/12; 435/7.1; 514/44; 514/561; 514/575; 514/557; 514/9

(57) ABSTRACT

The present invention relates to methods for identifying agents useful for alleviating insulin resistance in mammals, said methods being enabled by the finding that the insulin receptor substrate 1 (IRS-1) and histone deacetylase 2 (HDAC2) physically interact. By inhibition of the deacetylase activity in this complex, the insulin sensitivity can be restored.

### CROSS REFERENCE TO RELATED APPLICATIONS

This application claims priority from Swedish Patent Application No. 0202157-4, filed Jul. 9, 2002. The entire content of this prior application is incorporated herein by reference in its entirety.

### TECHNICAL FIELD

The present invention relates to methods for identifying agents useful for alleviating insulin resistance in mammals, said methods being enabled by the finding that the insulin receptor substrate 1 (IRS-1) and histone deacetylase 2 (HDAC2) physically interact. By inhibition of the deacetylase activity in this complex, the insulin sensitivity can be restored.

### BACKGROUND

The insulin receptor substrate proteins represent key elements in insulin and insulin-like growth factor (IGF) actions, transducing pleiotropic effects on cellular function and regulating processes such as metabolism, growth, cell differentiation and survival [1, 2]. At least four members (IRS 1-4) have been identified that differ as to tissue distribution, subcellular localization, developmental expression, binding to the insulin receptor, and interaction with Src homology 2 (SH2) domains (see below). They are all structurally characterized by N-terminal pleckstrin-homology and phosphotyrosine-binding domains, which are required for coupling to the activated insulin/IGF receptors, and a C-terminal region with multiple sites of tyrosine phosphorylation. IRS proteins thus act as molecular adapters in recruiting, inter alia, a number of SH2-containing proteins binding to specific phosphorylated tyrosine residues. This leads to activation of different intracellular cascades [2], one of which being the PI 3-kinase signaling cascade implicated in mediating the metabolic effects of insulin [3].

Targeted deletions of these proteins in mice have started to reveal some of their distinct physiological roles. Ablation of IRS-1 causes severe growth retardation with mild insulin resistance [4], [5], in contrast to ablation of IRS-2 which causes combined insulin resistance in peripheral tissues and impaired growth of β-cells [6]. Ablation of IRS-3 is devoid of a clear phenotype [7] whereas ablation of IRS-4 is associated with modest insulin resistance [8]. The available data are consistent with the notion that IRS-1 and IRS-2 are not functionally interchangeable in tissues that are responsible for glucose production (liver), glucose uptake (skeletal muscle and adipose tissue), and insulin production (pancreatic β-cells). In fact, IRS-1 appears to have its major role in skeletal muscle whereas IRS-2 appears to regulate hepatic insulin action as well as pancreatic β-cell development and survival.

IRS-1 was the first docking protein identified in mammalian systems [9]. The cDNA predicted a protein of 131 kDa but due to high serine/threonine phosphorylation it migrates on SDS-PAGE to a position corresponding to 165-180 kDa. IRS-1 contains 21 putative tyrosine phosphorylation sites, several of which are located in amino acid sequence motifs that bind to SH-2 domain proteins, including the p85 regulatory subunit of PI 3-kinase, Grb-2, Nck, crk, c-fyn, Csk, phospholipase Cγ and SHP-2 [3]. IRS-1 also contains more than 30 potential serine/threonine phosphorylation sites in motifs recognized by various kinases such as casein kinase II, protein kinase C, protein kinase B/Akt, and mitogen-activated protein (MAP) kinases [3]. It has been much discussed lately that increased serine phosphorylation of IRS-1 lowers its tyrosine phosphorylation by the insulin receptor and hence leads to insulin resistance [10].

It is known that in the transcription of genes the chromatin structure plays an important role and multiple signaling pathways converge on histones [11]. The covalent modifications of histone NH₂-tails that exist are acetylation, phosphorylation, and methylation. These post-transcriptional modifications affect the condensation status of the chromatin and hence regulate the access to the underlying DNA [12]. This “histone code” considerably extends the information potential of the genetic code [13].

Multiple histone acetyltransferases (HATs) and histone deacetylases (HDACs) control the state of chromatin acetylation and hence play a regulatory role in modulating the structure and function of chromatin [14, 15]. The acetyl-mediated signals are thus reversed by HDACs counteracting the effects of HATs by deacetylating lysine residues on histone tails. In higher eukaryotes, HDACs can be subdivided into three distinct groups known as classes I, II, III respectively, according to similarities of their sequences to those of yeast founding members [16]. To date, four enzymes, HDAC1, 2, 3, & 8 are the known members of class I deacetylases [15]. HDAC1 and HDAC2 (GenBank Accession No. XM_{—}004370) are the best characterized, and are chief constituents of the multiprotein transcriptional-repression complex Sin3/HDAC and the nucleosome remodelling deacetylase NuRD/Mi2/NRD complex [17]. Complexes that contain class I HDACs bind to numerous transcription factors, either directly, or indirectly through the nuclear-hormone corepressors NCOR and SMRT (silencing mediator for retinoid and thyroid hormone receptors). Although all class I and II HDACs can deacetylate histone tails, it seems that other cellular proteins can be specifically targeted by different HDACs as well [18].

A variety of different non-specific histone deacetylase inhibitors are known in the art [24, 25]. These fall into four broad categories including the butyrates, hydroxamic acids, benzamides and cyclic peptides (WO 02/06307, JP 01/348340, EP 1170008, WO 01/70675, WO 01/38322, WO 00/52033, WO 00/21979, JP 11302173, WO 99/11659, GB 2309696). Several are under investigation in clinical trials in humans. In particular, hydroxamic acids related to trichostatin A, such as suberoylanilide hydroxamic acid (SAHA) are well-tolerated, are not toxic and display biological activity [26].

### BRIEF DESCRIPTION OF THE DRAWINGS

FIG. 1 shows mapping of IRS-1/HDAC2 interaction site in the Matchmaker 3 yeast two-hybrid system.

FIG. 2 illustrates immunoprecipitation of IRS-1 and detection of anti-acetyl-lysine. MCF-7 cells were treated as follows: lane 1: IGF-1 for 10 min; lane 2: IGF-1 for 30 min; lane 3: IGF-1 for 1 h; lane 4: IGF-1 for 6 h; lane 5: IGF-1 for 24 h; lane 6: PMA for 6 h; lane 7: PMA for 6 h and IGF-1 for 10 min; lane 8: PMA for 24 h; lane 9: PMA and TSA for 6 h; lane 10: TSA for 6 h; lane 11: control (vehicle).

FIG. 3 illustrates immunoprecipitation of IRS-1 and detection of antiphosphotyrosine. MCF-7 cells were treated as follows: lane 1: insulin 10 min; lane 2: PMA & TSA for 4 h and insulin 10 min; lane 3: PMA for 4 h and insulin 10 min; lane 4: PMA & TSA for 4 h; lane 5: PMA for 4 h; lane 6: control (vehicle);

FIG. 4 illustrates coimmunoprecipitation of IRS-1 and HDAC2 in mouse liver tissue. Lane 1: ob/ob; lane 2: C57BL/6J; lane 3: PTP1B KO; lane 4: balb/cJJ.

### DISCLOSURE OF THE INVENTION

The present invention relates to the surprising finding that the insulin receptor substrate 1 (IRS-1) and histone deacetylase 2 (HDAC2) physically interact. By inhibition of the deacetylase activity in this complex in vivo, in different insulin resistant states, the insulin sensitivity can be restored, as seen as an increased tyrosine phosphorylation of IRS-1 by the insulin receptor.

Specifically, the inventors have found that IRS-1 and HDAC2 interact in the cytoplasmic compartment of yeast cells. The “Cytotrap” (Stratagene) yeast two-hybrid system enabled detection a novel interaction partner of IRS-1. The above-mentioned interaction was confirmed through coimmunoprecipitation of in vitro transcribed and translated IRS-1 and HDAC2 proteins. The interaction has been mapped to the C-terminal region of the IRS-1 molecule [19] and the C-terminal part of HDAC2.

Further, it has been found that IRS-1 is acetylated and that this post-translational modification can be enhanced by adding Trichostatin A (TSA) to MCF-7 cells. TSA is an inhibitor of class I and II histone deacetylases [18].

Animal models have been employed to test the hypothesis that the interaction between IRS-1 and HDAC2 is more prominent in an insulin resistant state. Liver tissue extracts from ob/ob mice (insulin resistant), PTP1B knock-out mice (insulin sensitized) and the corresponding control animals were investigated concerning the IRS-1/HDAC2 interaction. No interaction could be seen in livers from the insulin sensitized animals while the insulin resistant mice showed a pronounced interaction in liver.

Consequently, in a first aspect this invention provides a method for identifying an agent useful for alleviating insulin resistance in a mammal, said method comprising:

(i) contacting a candidate agent with a mammalian HDAC2 polypeptide or a mammalian HDAC2 polynucleotide; and

(ii) determining whether said candidate agent inhibits the biological activities of the said polypeptide or the expression of the said polynucleotide.

In one example, a method for identifying an agent that alleviates insulin resistance in a mammal includes the following steps: (i) contacting a candidate agent with a mammalian HDAC2 polypeptide or a mammalian HDAC2 polynucleotide; (ii) identifying the candidate agent as an inhibitor of a biological activity of the polypeptide or expression of the polynucleotide; and (iii) determining whether the candidate agent alleviates insulin resistance in a mammal. The method can also include a step of determining whether the candidate agent is effective in the treatment of type 2 diabetes.

In another example, a method for identifying an agent that alleviates insulin resistance in a mammal includes the following steps: (i) providing a candidate agent that inhibits a biological activity of a mammalian HDAC2 polypeptide or expression of a mammalian HDAC2 polynucleotide; and (ii) determining whether the candidate agent alleviates insulin resistance in a mammal. The method can also include a step of determining whether the candidate agent is effective in the treatment of type 2 diabetes.

In another aspect, the invention provides a method for identifying an agent useful for alleviating insulin resistance in a mammal, said method comprising:

(i) contacting a candidate agent with a mammalian IRS-1 polypeptide; and

(ii) determining whether said candidate agent increases acetylation of the said IRS-1 polypeptide. Optionally, the method could include additional steps, such as determining the state of chromatin acetylation or IRS-1 acetylation by action of histone acetyltransferases (HATs) and/or histone deacetylases (HDACs). The method can also include a step of determining whether the candidate agent is effective in alleviating insulin resistance or determining whether the candidate agent is effective in the treatment of type 2 diabetes.

A candidate agent can contain, for example, a peptide, peptidomimetic, amino acid, amino acid analog, polynucleotide, polynucleotide analog, nucleotide, nucleotide analog, or other small molecule. The said agent useful for alleviating insulin resistance can be a known HDAC inhibitor, such as a hydroxamic acid derivative such as trichostatin A, a cyclic tetrapeptide such as CHAP-31, a benzamide such as MS-27-275 or a butyrate such as phenyl butyrate. The said agent useful for alleviating insulin resistance is, in particular, useful for the treatment of type 2 diabetes mellitus, lipodystrophy-associated diabetes mellitus and pharmaceutical therapy-induced diabetes mellitus.

The methods described herein can be carried out in vitro or in vivo using a cell-based system, a cell-free system, or a combination of cell-based and cell-free systems.

In solution assays, methods of the invention comprise the steps of (a) contacting a HDAC2 or IRS-1 polypeptide with one or more candidate agents and (b) identifying the compounds that bind to the HDAC2 or IRS-1 polypeptide. Identification of the compounds that bind the HDAC2 polypeptide can be achieved by isolating the HDAC2 or IRS-1 polypeptide/binding partner complex, and separating the binding partner compound from the HDAC2 or IRS-1 polypeptide. An additional step of characterizing the physical, biological, and/or biochemical properties of the binding partner compound is also comprehended.

In one example, the invention includes a method for identifying an agent that alleviates insulin resistance in a mammal, the method including the following steps:

(i) contacting an HDAC2 polypeptide or an IRS-1 polypeptide with a candidate agent;

(ii) detecting the binding of the candidate agent to the HDAC2 polypeptide or the IRS-1 polypeptide; and (iii) determining whether the candidate agent alleviates insulin resistance in a mammal. The method can also include a step of determining whether the candidate agent is effective in the treatment of type 2 diabetes.

In one variation of an in vitro assay, the invention provides a method comprising the steps of (a) contacting an immobilized HDAC2 or IRS-1 polypeptide with a candidate binding partner compound and (b) detecting binding of the candidate compound to the HDAC2 or IRS-1 polypeptide. In an alternative embodiment, the candidate binding partner compound is immobilized and binding of HDAC2 or IRS-1 is detected. Immobilization is accomplished using any of the methods well known in the art, including covalent bonding to a support, a bead, or a chromatographic resin, as well as non-covalent, high affinity interactions such as antibody binding, or use of streptavidin/biotin binding wherein the immobilized compound includes a biotin moiety. Detection of binding can be accomplished (i) using a radioactive label on the compound that is not immobilized, (ii) using of a fluorescent label on the non-immobilized compound, (iii) using an antibody immunospecific for the non-immobilized compound, (iv) using a label on the non-immobilized compound that excites a fluorescent support to which the immobilized compound is attached, as well as other techniques well known and routinely practiced in the art.

The invention also provides cell-based assays to identify binding partner compounds of a HDAC2 or IRS-1 polypeptide. In one embodiment, the invention provides a method comprising the steps of contacting a HDAC2 or IRS-1 polypeptide expressed in a cell with a candidate binding partner compound and detecting binding of the candidate binding partner compound to the HDAC2 or IRS-1 polypeptide.

Binding of a candidate agent to a target polypeptide or polynucleotide can be determined by standard procedures which are well known in the art, including gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like.

A transfection assay can be a particularly useful screening assay for identifying an effective agent. In a transfection assay, a nucleic acid containing a gene such as a reporter gene that is operably linked to a HDAC2 promoter, a histone acetyl transferase promoter or an IRS-1 promoter, or an active fragment thereof, is transfected into the desired cell type. A test level of reporter gene expression is assayed in the presence of a candidate agent and compared to a control level of expression. An effective agent is identified as an agent that results in a test level of expression that is different than a control level of reporter gene expression, which is the level of expression determined in the absence of the agent. Methods for transfecting cells and a variety of convenient reporter genes are well known in the art (see, for example, Goeddel (ed.), Methods Enzymol., Vol. 185, San Diego: Academic Press, Inc. (1990); see also Sambrook, supra).

The invention also comprises a method for alleviating insulin resistance in a mammal, comprising administering to the mammal, including man, an effective amount of an inhibitor of HDAC2 and/or an agent increasing acetylation of IRS-1.

Another aspect of the invention is a pharmaceutical formulation, for use in the treatment or prevention of insulin resistance, wherein the active ingredient is an inhibitor of HDAC2; and/or an agent increasing acetylation of IRS-1.

Throughout this description the terms “standard protocols” and “standard procedures”, when used in the context of molecular biology techniques, are to be understood as protocols and procedures found in an ordinary laboratory manual such as: Current Protocols in Molecular Biology, editors F. Ausubel et al., John Wiley and Sons, Inc. 1994, or Sambrook, J., Fritsch, E. F. and Maniatis, T., Molecular Cloning: A laboratory manual, 2nd Ed., Cold Spring Harbor Laboratory Press, Cold Spring Harbor, N.Y. 1989.

Unless otherwise defined, all technical and scientific terms used herein have the same meaning as commonly understood by one of ordinary skill in the art to which this invention belongs. Suitable methods and materials are described below, although methods and materials similar or equivalent to those described herein can also be used in the practice or testing of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

The invention will now be further illustrated through the description of examples of its practice. The examples are not intended as limiting in any way of the scope of the invention.

### EXAMPLES

### Example 1

### Detection of the IRS-1/HDAC2 Protein-protein Interaction in the Cytotrap™ Yeast Two-hybrid System

The Cytotrap™ yeast two-hybrid system was used to discover protein-protein interactions in the cytoplasm of yeast cells. Interactions were detected by recruitment of the human Sos (hSos) gene product to the cell membrane, which activates the Ras pathway. The yeast strain used (cdc25H) harbors a temperature sensitive mutation in the cdc25 gene, the yeast homologue for hSos, which means that the cells can grow at 25° C. but not at 37° C. unless rescued with a protein-protein interaction.

A human fetal brain plasmid cDNA library (Stratagene), harbored in the pMyr vector (with a myristylation signal to direct and anchor proteins in the membrane), was used as “prey” and the subcloned full length IRS-1 gene in the pSos vector was used as “bait”. When prey and bait proteins interact the hSos is brought into close proximity to Ras and subsequently the yeast survive and are selected by growth at 37° C. The IRS-1/HDAC2 interaction rescued growth at 37° C. in this way. The corresponding pMyr yeast plasmid was isolated and cotransformed with the pSos bait construct to perform false positive testings. The results showed that growth on galactose media at 37° C. was dependent on the IRS-1/HDAC2 interaction. With glucose media, no growth could be seen (the GAL1 promoter of the pMyr vector not induced and no unspecific interaction between bait and Lamin C (pMyrLamC) appeared.

### Example 2

### Confirmation of the Two-hybrid Protein-protein Interaction Through in vitro Coimmunoprecipitation of in vitro Transcribed and Translated Proteins

In order to confirm the IRS-1/HDAC2 interaction in vitro, a coupled transcription/translation system (Promega) was used. This is a method that combines a rabbit reticulocyte lysate solution with RNA polymerase, nucleotides, salts, a ribonucleoside inhibitor, and [³⁵S]-methionine to allow detection of translated proteins. Since the prey vector pMyr already contains a T7 promoter, this can be used directly in the system. The bait vector pSos on the other hand lacks a T7 promoter and thus the IRS-1 gene had to be subcloned into a T7-containing vector (pGBKT7) to permit transcription. The individually transcribed and translated proteins were mixed and coimmunoprecipitated with anti-IRS-1 antibodies and subsequently analyzed with polyacrylamide gel electrophoresis (4-12%). The gel was dried down and the incorporated [³⁵S]-methionine enabled analysis with a phosphorimager. Both protein bands (IRS-1 and HDAC2) showed up in the same lane and had therefore been pulled down together by the IRS-1 antibody. The HDAC2 band did not correspond to full-length protein but to the C-terminal, truncated part found in the yeast two-hybrid screen (˜31 kDa).

### Example 3

### Coimmunoprecipitations of IRS-1 and HDAC2 in Mammalian Cells

To further validate the IRS-1/HDAC2 interaction, several mammalian cell lines were used. MCF-7 is a human breast adenocarcinoma cell line, HepG2 is a hepatocellular carcinoma cell line and L6 is a rat skeletal muscle cell line. The MCF-7 cell line was chosen since these cells have a high endogenous production of IRS-1. Cells were grown to confluency, treated with IGF-1 or PMA (phorbol myristic acid) for different lengths of time and then harvested in hypotonic cell lysis buffer (comprising 20 mM Hepes, pH 7.6, 20% glycerol, 10 mM NaCl, 1.5 mM MgCl₂, 0.2 mM EDTA, 0.1% NP40, 25 mM NaF, 25 mM β-glycerophosphate, 1 mM DTT, 1 mM Na orthovanadate with protease inhibitors). All precleared fractions were matched for protein content and then immunoprecipitated with anti-IRS-1 antibody. The precipitates were resolved by polyacrylamide gel electrophoresis (PAGE 4-12%), blotted to a membrane and subsequently probed with anti-HDAC2 antibody plus the appropriate HRP-conjugated secondary antibody (protein bands revealed with ECL-detection). The results demonstrated that the interaction is PMA-driven (PMA activates protein kinase C isoforms which is known to make the cells insulin resistant). A similar. experiment was performed the other way around, i.e. HDAC2 was immunoprecipitated from the cell extracts and the subsequent membrane was probed with anti-IRS-1 antibodies. The results are consistent, showing that the interaction is PMA-driven. In the use of L6 and HepG2 cells, the interaction between IRS-1 and HDAC2, which was not influenced by stimulation with insulin, was confirmed.

### Example 4

### Mapping the Interaction Site of HDAC2 on IRS-1

HDAC2 was full length cloned using RACE cDNA obtained from human heart tissue together with gene specific primers. With the purpose of mapping the interaction site of HDAC2 on IRS-1, the Matchmaker-3™ yeast two-hybrid system (Clontech) was used. This is a GAL4-based two-hybrid system that provides a transcriptional assay for detecting specific protein-protein interactions in yeast. Two nutritional markers and one enzymatic reporter gene are used to detect interactions.

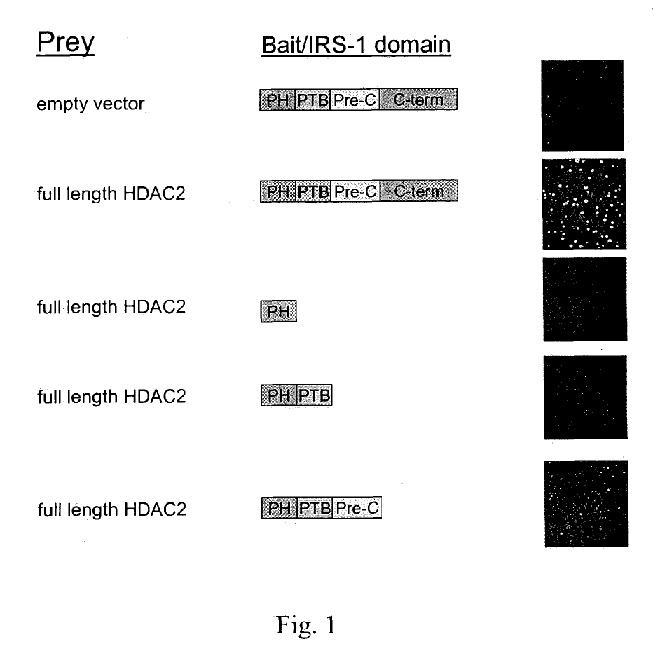

Different domains of IRS-1 (obtained by PCR) were subcloned into a “bait” vector pGBKT7), fused to the DNA-binding domain of GAL4. Full length HDAC2 was subcloned into the “prey” vector (pGADT7), fused to the activation domain of GAL4. Provided that bait and prey interact, the reporter genes are turned on and yeast cells can grow on media lacking the two nutritional markers. The results (FIG. 1) showed that the interaction takes place between the pre C-terminal region of IRS-1 and the C-terminal part of HDAC2, as judged from the original clone.

### Example 5

### IRS-1 is Post-translationally Acetylated

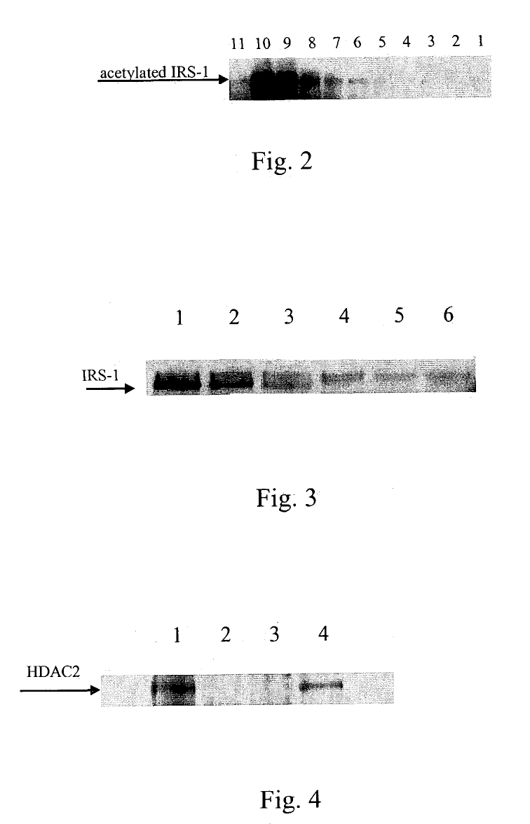

The interaction between IRS-1 and HDAC2 suggested that IRS-1 may be acetylated, a modification that could be regulated by HDAC2. This was tested by Western blotting, using an anti-acetyl-lysine antibody. MCF-7 cells, grown to confluency, were treated with IGF-1, PMA or TSA for different lengths of time. Cells were harvested as described in Example 3. Following protein determinations, immunoprecipitation with anti-IRS-1 antibodies and western blot with anti-acetyl lysine antibodies, a basal acetylation of IRS-1 could be seen, which was significantly pronounced in fractions that had been treated with TSA (an HDAC inhibitor; FIG. 2). Hence, a consequence of inhibiting the deacetylase that binds to IRS-1 is that IRS-1 becomes heavily acetylated.

### Example 6

### Reversal of Insulin Resistance by Inhibition of HDAC

To address the question whether the interaction between IRS-1 and HDAC2 affects the tyrosine phosphorylation status of IRS-1, a series of cell experiments was performed. MCF-7 cells (ATCC Accession No. HTB-22), grown to confluency, were treated with phorbol myristic acid (PMA) (4 h), TSA (4 h) and insulin (10 min). Cells were harvested as described in Example 3. The different fractions were matched for protein content and subsequently immunoprecipitated with anti-IRS-1 antibodies. The precipitates were electrophoresed and western blotted with anti-phospho-tyrosine antibodies. The results clearly showed that PMA makes the cells insulin resistant (a lesser degree of tyrosine phosphorylation in lane 4 than in lane 1; FIG. 3). The results also indicated that when the cells are simultaneously treated with TSA, insulin resistance can to a large extent be remedied. Thus, inhibition of HDAC2, which thereby increases the acetylation level of IRS-1, significantly facilitates tyrosine phosphorylation of the IRS-1 molecule.

### Example 7

### The IRS-1/HDAC2 Interaction is Seen in Animal Models of Insulin Resistance But Not in Models of Insulin Sensitivity

To ascertain whereas the IRS-1/HDAC2 interaction is valid also in animal tissue, different mouse strains were investigated. The leptin-deficient ob/ob mouse model is characterized by morbid obesity (the C57BL/6J genetic background; [21]). These mice are insulin resistant and thus serve as a model of diabetes. A mouse model at the other extreme is the PTPIB knock-out mouse (balb/cJJ genetic background; [22]), with enhanced insulin sensitivity due to increased phosphorylation of the insulin receptor in muscle and liver tissue.

Liver tissue from ob/ob mice, PTPIB knock-out mice and the corresponding controls was powdered using a pestle and mortar (pre-cooled to −80° C.) and subsequently homogenized at 4° C. using a Polytron. For every gram of tissue, 3 ml of homogenization buffer (comprising 4.0 mM EDTA, 50.0 mM NaF pH 8.0, 1.0 mM Na-orthovanadate, 1.0 μM ocadaic acid, 0.1% 2-mercaptoethanol and proteinase inhibitor cocktail; [23]) were used. The homogenate was centrifuged at 13,000xg for 10 minutes at 4° C. whereafter the supernatant was used immediately or snap-frozen in liquid nitrogen. Liver extracts from the four different mouse strains were matched for protein content and immunoprecipitated with anti-IRS-1 antibodies. The following Western blot was performed as described in Example 3, with the use of anti-HDAC2 antibodies to detect the level of interaction between IRS-1 and HDAC2. Results (FIG. 4) showed that in the balb/c genetic background IRS-1 and HDAC2 interact, but that this interaction disappears in the insulin-sensitized animal (PTP1B KO). By contrast, in the insulin resistant animal (ob/ob) the interaction is visible and clear while no coimmunoprecipitation can be seen in the respective control animal. This leads us to conclude that the IRS-1/HDAC2 interaction correlates with reduced sensitivity to insulin which may be associated with increased serine phosphorylation of IRS-1.

References

1. White, M. F., R. Maron, and C. R. Kahn, Insulin rapidly stimulates tyrosine phosphorylation of a Mr-185,000 protein in intact cells. Nature, 1985. 318(6042): p. 183-6.

2. White, M. F., The IRS-signalling system: a network of docking proteins that mediate insulin action. Mol Cell Biochem, 1998. 182(1-2): p. 3-11.

3. Shepherd, P. R., D. J. Withers, and K. Siddle, Phosphoinositide 3-kinase: the key switch mechanism in insulin signalling. Biochem J, 1998. 333(Pt 3): p. 471-90.

4. Araki, E., et al., Alternative pathway of insulin signalling in mice with targeted disruption of the IRS-1 gene. Nature, 1994. 372(6502): p. 186-90.

5. Tamemoto, H., et al., Insulin resistance and growth retardation in mice lacking insulin receptor substrate-1. Nature, 1994. 372(6502): p. 182-6.

6. Withers, D. J., et al., Disruption of IRS-2 causes type 2 diabetes in mice. Nature, 1998. 391(6670): p. 900-4.

7. Liu, S. C., et al., Insulin receptor substrate 3 is not essential for growth or glucose homeostasis. J Biol Chem, 1999. 274(25): p. 18093-9.

8. Fantin, V. R., et al., Mice lacking insulin receptor substrate 4 exhibit mild defects in growth, reproduction, and glucose homeostasis. Am J Physiol Endocrinol Metab, 2000. 278(1): p. E127-33.

9. Sun, X. J., et al., Structure of the insulin receptor substrate IRS-1 defines a unique signal transduction protein. Nature, 1991. 352(6330): p. 73-7.

10. Qiao, L. Y., et al., Identification of enhanced serine kinase activity in insulin resistance. J Biol Chem, 1999. 274(15): p. 10625-32.

11. Cheung, P., C. D. Allis, and P. Sassone-Corsi, Signaling to chromatin through histone modifications. Cell, 2000. 103(2): p. 263-71.

12. Wolffe, A. P. and D. Guschin, Review: chromatin structural features and targets that regulate transcription. J Struct Biol, 2000. 129(2-3): p. 102-22.

13. Strahl, B. D. and C. D. Allis, The language of covalent histone modifications. Nature, 2000. 403(6765): p. 41-5.

14. Roth, S. Y., J. M. Denu, and C. D. Allis, Histone acetyltransferases. Annu Rev Biochem, 2001. 70: p. 81-120.

15. Ng, H. H. and A. Bird, Histone deacetylases: silencers for hire. Trends Biochem Sci, 2000. 25(3): p. 121-6.

16. Gray, S. G. and T. J. Ekstrom, The human histone deacetylase family. Exp Cell Res, 2001. 262(2): p. 75-83.

17. Knoepfler, P. S. and R. N. Eisenman, Sin meets NuRD and other tails of repression. Cell, 1999. 99(5): p. 447-50.

18. Vigushin, D. M. and R. C. Coombes, Histone deacetylase inhibitors in cancer treatment. Anticancer Drugs, 2002. 13(1): p. 1-13.

19. Tu, Y., et al., Src homology 3 domain-dependent interaction of Nck-2 with insulin receptor substrate-1. Biochem J, 2001. 354(Pt 2): p. 315-22.

20. [deleted]

21. Ewart-Toland, A., et al., Effect of the genetic background on the reproduction of leptin-deficient obese mice. Endocrinology, 1999. 140(2): p. 732-8.

22. Elchebly, M., et al., Increased insulin sensitivity and obesity resistance in mice lacking the protein tyrosine phosphatase-1B gene. Science, 1999. 283(5407): p. 1544-8.

23. Cross, D. A., et al., Insulin activates protein kinase B. inhibits glycogen synthase kinase-3 and activates glycogen synthase by rapamycin-insensitive pathways in skeletal muscle and adipose tissue. FEBS Lett, 1997. 406(1-2): p. 211-5.

24. Johnstone, R. W. Histone deacetylase inhibitors: novel drugs for the treatment of cancer. Nature Reviews, 2002. 1: p. 287-299.

25. Marks, P. A et al., Histone deacetylase inhibitors as new cancer drugs. Current Opinion in Oncology, 2001. 13: p. 477-83.

26. Kelly, W. K. et al., Suberoylanilide hydroxamic acid (SAHA), a histone deacetylase inhibitor: biologic activity without toxicity. Proc. Amer. Soc. Clin. Oncol., 2001. 20: p. 87a.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method for identifying an agent that alleviates insulin resistance in a mammal, the method comprising:
contacting a candidate agent with a mammalian histone deacetylase 2 (HDAC2) polypeptide or a mammalian HDAC2 polynucleotide;
identifying the candidate agent as an inhibitor of a biological activity of the polypeptide or expression of the polynucleotide; and
determining whether the candidate agent alleviates insulin resistance in a mammal.

2. The method of claim 1, wherein the candidate agent is a peptide, peptidomimetic, amino acid, amino acid analog, polynucleotide, polynucleotide analog, nucleotide, or nucleotide analog.

3. The method of claim 1, wherein the candidate agent is a hydroxamic acid derivative, cyclic tetrapeptide, benzamide, or butyrate.

4. The method of claim 1, wherein the candidate agent inhibits deacetylase activity of HDAC2.

5. The method of claim 1, comprising determining whether the candidate agent is effective in the treatment of type 2 diabetes.

6. A method for identifying an agent that alleviates insulin resistance in a mammal, the method comprising:
providing a candidate agent that inhibits a biological activity of a mammalian HDAC2 polypeptide or expression of a mammalian HDAC2 polynucleotide; and
determining whether the candidate agent alleviates insulin resistance in a mammal.

7. The method of claim 6, wherein the candidate agent is a peptide, peptidomimetic, amino acid, amino acid analog, polynucleotide, polynucleotide analog, nucleotide, or nucleotide analog.

8. The method of claim 6, wherein the candidate agent is a hydroxamic acid derivative, cyclic tetrapeptide, benzamide, or butyrate.

9. The method of claim 6, wherein the candidate agent inhibits deacetylase activity of HDAC2.

10. The method of claim 6, comprising determining whether the candidate agent is effective in the treatment of type 2 diabetes.

11. A method for identifying an agent that alleviates insulin resistance in a mammal, the method comprising:
contacting an HDAC2 polypeptide or an insulin receptor substrate 1 (IRS-1) polypeptide with a candidate agent;
detecting the binding of the candidate agent to the HDAC2 polypeptide or the IRS-1 polypeptide; and
determining whether the candidate agent alleviates insulin resistance in a mammal.

12. The method of claim 11, wherein the HDAC2 polypeptide or the IRS-polypeptide is immobilized during the contacting step.

13. The method of claim 11, wherein the candidate agent is immobilized during the contacting step.

14. The method of claim 11, wherein the candidate agent is a peptide, peptidomimetic, amino acid, amino acid analog, polynucleotide, polynucleotide analog, nucleotide, or nucleotide analog.

15. The method of claim 11, wherein the contacting step is carried out using an in vitro system.

16. The method of claim 15, wherein the in vitro system is a cell-free system.

17. The method of claim 11, further comprising determining whether the candidate agent is effective in the treatment of type 2 diabetes.

18. A method for identifying an agent that increases acetylation of IRS-1, the method comprising:
contacting a candidate agent with a mammalian IRS-1 polypeptide; and
determining whether the candidate agent increases acetylation of the IRS-1 polypeptide.

19. The method of claim 18, further comprising determining whether the candidate agent is effective in alleviating insulin resistance.

20. The method of claim 18, further comprising determining whether the candidate agent is effective in the treatment of type 2 diabetes.

21. A method for alleviating insulin resistance in a mammal, the method comprising administering to a mammal in need thereof an effective amount of an inhibitor of HDAC2.

22. The method according to claim 21, wherein the inhibitor of HDAC2 is trichostatin A.

23. The method of claim 21, wherein the inhibitor of HDAC2 is a hydroxamic acid derivative, cyclic tetrapeptide, benzamide, or butyrate.

24. The method of claim 21, wherein the inhibitor of HDAC2 inhibits deacetylase activity of HDAC2.

25. The method of claim 21, wherein the mammal is a human.

26. The method of claim 25, wherein the human has type 2 diabetes.

27. A method for alleviating insulin resistance in a mammal, the method comprising administering to a mammal in need thereof an effective amount of an agent that increases acetylation of IRS-1.

28. The method of claim 27, wherein the mammal is a human.

29. The method of claim 28, wherein the human has type 2 diabetes.
